(19) United States
(12) Patent Application Publication
(10) Pub. No.: US 2023/0380766 A1
(43) Pub. Date: Nov. 30, 2023

(54) ENDOVASCULAR ELECTROENCEPHALOGRAPHY (EEG) AND ELECTROCORTICOGRAPHY (ECoG) DEVICES, SYSTEMS AND METHODS

(71) Applicant: PRECISION NEUROSCIENCE CORPORATION, New York, NY (US)

(72) Inventor: Benjamin I. RAPOPORT, New York, NY (US)

(73) Assignee: PRECISION NEUROSCIENCE CORPORATION, New York, NY (US)

(21) Appl. No.: 18/230,998

(22) Filed: Aug. 7, 2023

Related U.S. Application Data
(63) Continuation of application No. 17/671,715, filed on Feb. 15, 2022.
Application No. 17/671,715 is a continuation of application No. 16/816,217, filed on Mar. 11, 2020.
(60) Provisional application No. 62/816,361, filed on Mar. 11, 2019.

(51) Int. Cl. A61B 5/00; A61N 1/05; A61L 31/02; A61B 5/24; A61B 5/291; A61B 5/293

(52) U.S. Cl. CPC A61B 5/6868; A61N 1/0529; A61L 31/022; A61B 5/6876; A61B 5/4836; A61B 5/6852; A61B 5/24; A61B 5/291; A61N 1/05; A61B 5/293; A61B 2017/00345

(57) ABSTRACT

The present disclosure is directed to systems and methods for endovascular electroencephalography (EEG) and electrocorticography (ECoG) systems. In some embodiments, the disclosed systems include electrode arrays that are configured to record and/or stimulate brain tissue via placement within blood vessels of the brain. Venous and arterial EEG and ECoG electrodes, ambulatory EEG and ECoG systems, and transcutaneous access and signal control systems for general and ambulatory endovascular electroencephalography (EEG) and electrocorticography (ECoG), as well as endovascular neural stimulating electrodes are discussed.

### CROSS-REFERENCE TO RELATED APPLICATIONS

The present disclosure is a continuation of U.S. application Ser. No. 17/671,715, titled ENDOVASCULAR ELECTROENCEPHALOGRAPHY (EEG) AND ELECTROCORTICOGRAPHY (ECoG) DEVICES, SYSTEMS AND METHODS, filed Feb. 15, 2002, which is a continuation of U.S. application Ser. No. 16/816,217, titled ENDOVASCULAR ELECTROENCEPHALOGRAPHY (EEG) AND ELECTROCORTICOGRAPHY (ECoG) DEVICES, SYSTEMS AND METHODS, filed Mar. 11, 2020, which claims priority to and the benefit of U.S. Provisional Application No. 62/816,361, titled ENDOVASCULAR ELECTROPHYSIOLOGY (EEG) SYSTEMS, AND RELATED SYSTEMS, APPARATUS, AND METHODS, filed on Mar. 11, 2019, the contents of which are hereby incorporated by reference.

This application is related to U.S. application Ser. No. 16/816,237, titled INTRADURAL NEURAL ELECTRODES, filed on Mar. 11, 2020, the contents of which are hereby incorporated by reference.

### TECHNICAL FIELD

The present disclosure is related to endovascular techniques for electroencephalography, electrocorticography, neural recording and stimulation, and more particularly, applications to ambulatory endovascular electroencephalography (EEG) and electrocorticography (ECoG).

### BACKGROUND

Several common disorders of the brain, spinal cord, and peripheral nervous system arise due to abnormal electrical activity in biological (neural) circuits. In general terms, these conditions may be classified into: (1) conditions such as epilepsy, in which electrical activity is dysregulated, and recurrent activity persists in an uncontrolled fashion; (2) conditions such as stroke or traumatic injury, in which an electrical pathway is disrupted, disconnecting a component of a functional neural circuit; and (3) conditions such as Parkinson's disease, in which neurons in a discrete region cease to function, leading to functional impairment in the neural circuits to which the lost neurons belong.

When the electrical lesion is focal and relatively discrete the effective diagnosis and treatment of such conditions depends on precise localization of the lesion and, when possible, restoration of normal electrophysiologic function to the affected region.

Conventional techniques for localizing electrical lesions in the brain such as imaging techniques, electromagnetic recording techniques, electrocorticography (ECoG), depth electrodes, and deep brain stimulation techniques, each have specific limitations.

For example, imaging techniques such as magnetic resonance imaging (Mill) and computed tomography (CT) are non-invasive methods of examining brain tissue. These imaging techniques may be useful in detecting and localizing functional lesions, including strokes, anatomic abnormalities capable of causing seizures, and foci of neuronal degeneration. However, not all functional lesions can be detected using these imaging modalities because these techniques do not image electrical activity. Furthermore, these imaging techniques lack temporal resolution, and provide no mechanism for therapeutic electrophysiologic intervention.

Electromagnetic recording techniques such as electroencephalography (EEG) and magnetoencephalography (MEG) are also noninvasive techniques. EEG and MEG are able to provide temporal resolution of electrical activity in the brain, and thus often used for seizure detection. In conventional EEG, electrodes are positioned on the scalp. However, the spatial resolution of electromagnetic recording techniques is limited, both due to physical distance of electrodes from the brain, and by the dielectric properties of scalp and skull. Accordingly, the spatial resolution of EEG is better for superficial regions, and worse for neural activity deep within the brain. For example, seizures arising from anatomic abnormalities near the cortical surface are well localized by EEG and MEG.

Electrocorticography (ECoG), or intracranial EEG, is a form of electroencephalography that provides improved spatial resolution by placing recording electrodes directly on the cortical surface of the brain (in contrast to conventional EEG systems where electrodes are positioned on the scalp). ECoG is frequently used during neurosurgical procedures to map normal brain function and locate abnormal electrical activity. However, ECoG requires a craniotomy or a temporary surgical removal of a significant portion of the skull, in order to expose the brain surfaces of interest. This exposes patients to the attendant risks of brain surgery. Furthermore, while electrical activity near the cortical surface of the brain can be mapped with reasonable spatial resolution, electrical activity deep within the brain remains difficult to localize using ECoG.

“Depth electrodes” record electrical activity with high spatial and temporal precision. However, depth electrodes are configured to record only from small volumes of tissue (i.e., small populations of neurons). Further, the placement of depth electrodes requires the disruption of normal brain tissue along the trajectory of the electrode, resulting in irreversible damage or destruction of some neurons. As such, depth electrodes are conventionally placed surgically, in a hypothesis-driven manner, and the number of such electrodes that can be safely placed simultaneously is limited. Further, this and other related techniques are static in that electrode positions cannot be adjusted once the electrodes are placed, except for small adjustments (to depth, in the case of depth electrodes) at the time of placement.

Deep brain stimulation (DBS) electrodes, a stimulating analog of recording depth electrodes, electrically stimulate brain regions with millimetric and/or sub-millimetric precision. They are implanted using minimally invasive surgical techniques, and can be effective in conditions such as Parkinson's disease and essential tremor, in which neuronal dysfunction is confined to small, discrete, and unambiguous regions of the brain. Some evidence suggests these techniques can be useful in treating epilepsy, as well as other disorders (not all of which are traditionally associated with focal brain lesions), including some psychiatric disorders and substance addiction. For example, symptoms of Parkinson's disease, arising from degeneration of dopamine-producing neurons in a well-defined region (the substantia nigra), can often be effectively modulated by precise stimulation of a millimetric nucleus (the subthalamic nucleus) using a small number of deep brain stimulation (DBS) electrodes.

Neural recording and stimulation techniques (including those discussed above) involve design trade-offs among a number of primary factors: (1) spatial resolution, (2) temporal resolution, (3) degree of invasiveness and collateral damage to normal brain tissue, and (4) optimization for electrical recording and/or electrical stimulation. An ideal electrophysiologic neural probe, should simultaneously provide optimal performance in all four of the above categories.

Diagnosis and treatment of functional electrophysiologic lesions in many brain regions remain challenging or intractable. In particular, deep brain regions are frequent sites of functional lesions, yet remain difficult to access systematically and minimally invasively. For example, the medial temporal lobe is a common site for seizure foci and the substantia nigra is the site of neuronal degeneration causing Parkinson's disease; both regions are several centimeters deep to the cortical surface. Accordingly, the conventional techniques discussed above such as imaging techniques, electromagnetic recording, ECoG, depth electrodes, or deep brain stimulation are ill- or imperfectly equipped to detect, localize, and treat these lesions in the brain.

### SUMMARY

The present disclosure is generally directed towards an endovascular EEG and ECoG system that provides improved spatial resolution, improved temporal resolution, lower degrees of invasiveness and collateral damage to normal brain tissue, and is capable of being optimized for electrical recording and/or electrical stimulation. In some embodiments, the endovascular EEG/ECoG system may be used as an ambulatory EEG/ECoG system.

The present disclosure relates to the electrophysiologic recording and stimulation of brain tissue using electrode arrays deployed within blood vessels.

In some embodiments, a catheter assembly is configured for insertion into a blood vessel of a head or brain, and includes a catheter and an electrode array comprising one or more electrodes configured to record or stimulate electrical activity in brain tissue, where the electrode array is positioned about the exterior surface of the catheter. In some embodiments, wires connected to the one or more electrodes are configured to traverse the length of the catheter. In some embodiments the electrodes include at least one of gold, silver, platinum, or platinum-iridium. In some embodiments, the electrodes have a diameter between about 5 to 25 microns. In some embodiments, the electrode array further includes an electrode array substrate comprising at least one of nitinol, polymer and/or polyether ether ketone (PEEK). In some embodiments the electrode array is connected via one or more wired connectors to a transcutaneous connector to an externally wearable computer unit. In some embodiments the electrode array is connected via one or more wired connectors to a subcutaneous connector to a subcutaneously implanted computer unit.

In some embodiments, an implantable medical device includes an expandable stent configured for insertion into a blood vessel of a head or brain, the expandable stent capable of transitioning between a collapsed configuration and an expanded configuration; and an electrode array including one or more electrodes configured to record or stimulate electrical activity in brain tissue, wherein the electrode array is positioned on the expandable stent.

Optionally, each of the electrodes includes at least one of gold, silver, platinum, or platinum-iridium, has a diameter between about 5 to 25 microns. The expandable stent may include an electrode array substrate comprising at least one of nitinol, polymer and/or polyether ether ketone (PEEK). In some embodiments, the electrode array may be connected via one or more wired connectors to a transcutaneous connector to an externally wearable computer unit. Optionally, the electrode array is connected via one or more wired connectors to a subcutaneous connector to a subcutaneously implanted computer unit.

In some embodiments, the implantable medical device is positioned within a blood vessel such as a dural venous sinus (including the superior sagittal sinus, transverse sinus, sigmoid sinus, or straight sinus), a superficial cortical vein, a deep cerebral vein or a tributary to any such vein, other cerebral veins, a branch of one of the internal carotid arteries, an artery of the posterior intracranial circulation, the vertebral artery or one of its branches, the basilar artery or one of its branches, the posterior cerebral artery or one of its branches, or a branch of the external carotid artery.

In some embodiments, the electrode array may be repositioned in the blood vessel after deployment.

In some embodiments, the electrode array can be collapsed and retrieved from the blood vessel and has a diameter between about 4 mm to about 12 mm and a length between about 20 to about 60 mm. In some embodiments the plurality of electrodes are fabricated on the electrode array scaffold using lithography, 3D printing, electroplating, or a covalent-type bonding process. In some embodiments the collapsible stent is cylindrical in shape. In some embodiments the collapsible stent has at least one tapered end.

In some embodiments a method includes the steps of advancing an endovascular catheter to access a blood vessel in the vascular system of a user, deploying an electrode array via the catheter, the electrode array comprising a substrate formed of at least one of nitinol, polymer and/or polyether ether ketone (PEEK) and a plurality of electrodes, by expanding a collapsible stent comprising the electrode array, positioning the electrode array within the blood vessel adjacent to a brain tissue, and recording or stimulating the brain tissue adjacent to the blood vessel. Further, the method may include the steps of recapturing the deployed electrode array by pulling either the endovascular catheter or one or more wires of the electrode array so as to collapse the collapsible stent and resheath the electrode array within the catheter, and removing the recaptured electrode array from the body, or recapturing the array by advancing a catheter over a wire of the array. Electrodes may be formed of at least one of gold, silver, platinum, or platinum-iridium. Each electrode may have a diameter between about 5 to 25 microns. The target blood vessel may be at least one of the dural venous sinus, the superior sagittal sinus, transverse sinus, sigmoid sinus straight sinus, superficial cortical vein, deep cerebral vein or a tributary to any such vein, cerebral veins, a branch of one of the internal carotid arteries, an artery of the posterior intracranial circulation, the vertebral artery or one of its branches, the basilar artery or one of its branches, the posterior cerebral artery or one of its branches, and a branch of the external carotid artery. In some embodiments, the method further includes the step of repositioning the electrode array within the blood vessel after it has been deployed.

### BRIEF DESCRIPTION OF THE DRAWINGS

FIG. 1A is a schematic diagram of a system built in accordance with embodiments of the present disclosure.

FIG. 1B is a flowchart illustrating a method in accordance with embodiments of the present disclosure.

FIG. 2 is a schematic diagram of an electrode array built in accordance with embodiments of the present disclosure.

FIG. 3 is a schematic diagram of an electrode array built in accordance with embodiments of the present disclosure.

FIG. 4A is a schematic diagram of an electrode array built in accordance with embodiments of the present disclosure in an expanded configuration.

FIG. 4B is a schematic diagram of an electrode array built in accordance with embodiments of the present disclosure in a collapsed configuration.

FIG. 5 is a schematic diagram of an electrode array built in accordance with embodiments of the present disclosure.

FIG. 6 is a schematic diagram of an electrode array built in accordance with embodiments of the present disclosure.

FIG. 7 is a schematic diagram of an electrode array built in accordance with embodiments of the present disclosure positioned within a blood vessel.

### DETAILED DESCRIPTION

The present disclosure is generally directed towards an endovascular EEG/ECoG system that provides improved spatial resolution, improved temporal resolution, lower degrees of invasiveness and collateral damage to normal brain tissue, and is capable of being optimized for electrical recording and/or electrical stimulation. In some embodiments, the endovascular EEG/ECoG system may be used as an ambulatory EEG/ECoG system.

The disclosed systems and methods may be used for neuro-electrophysiology, mapping (recording) and stimulation of the brain and nervous system to diagnose and treat a variety of conditions including epilepsy/seizure disorders, conditions such as paralysis associated with stroke or spinal cord injury; movement disorders such as Parkinson's disease and essential tremor; chronic pain disorders; neuro-endocrine disorders (including disorders traditionally associated with the hypothalamic-pituitary system as well as disorders such as obesity, which have hypothalamic components); and human-to-computer interfaces. In some embodiments, the disclosed systems and methods may include implants that are configured to be implanted for minutes to hours, or days to weeks, for diagnostic procedures, inpatient monitoring, and/or outpatient monitoring. Depending on the target area of the brain, specific arteries and veins may be used for peripheral access. The disclosed systems and methods may include any combination of catheter-based, wire-based, and/or stent-based electrodes. The disclosed systems and methods may be used for recording only, stimulation only, and/or both.

For example, in some embodiments, an endovascular EEG/ECoG may be used for medium-term recording, where an EEG/ECoG is implanted in an outpatient procedure for several days to weeks. Electrodes may be reversibly implanted in the brain, and have wires that are tunneled to a subclavian or upper extremity or lower extremity or other venous access port and then connected via a transcutaneous connector to an external wearable computer. In some embodiments, the electrodes may be located or configured in a self-expandable stent that is placed in a vascular location such as the lateral (transverse or sigmoid) venous sinus. Access to the brain may be by the axillary, basilic, cephalic, subclavian, or other veins. Transcutaneous connectors (leads or electrodes) may be used to connect the endovascular EEG/ECoG components to an external wearable device. In some embodiments, an endovascular EEG/ECoG may be configured to record and/or stimulate for up to a month or even longer. An endovascular EEG/ECoG may include a stent having an unconstrained diameter between about 3-10 mm, and a length between 30-40 mm.

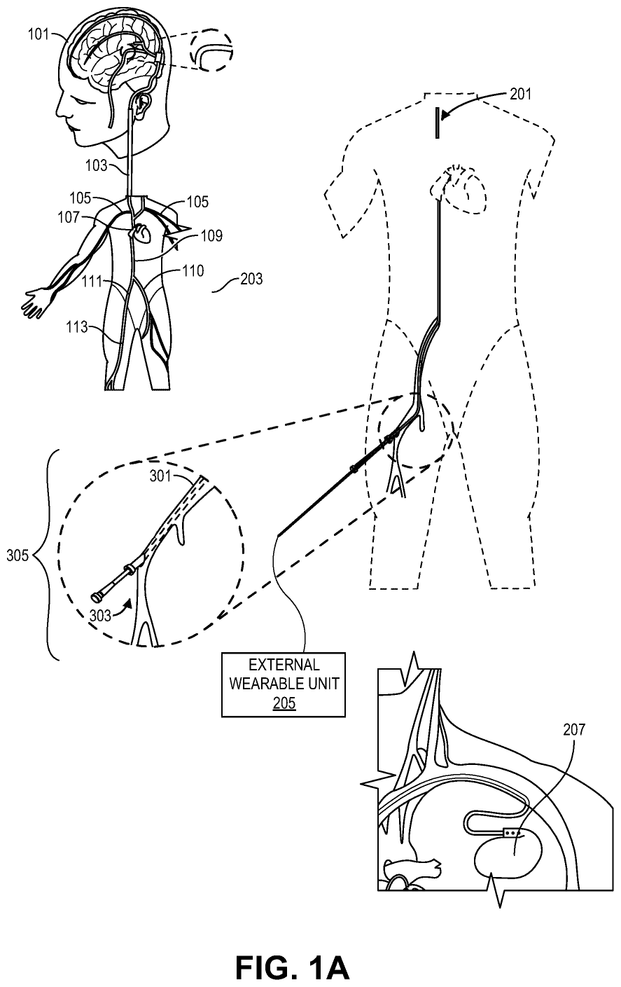

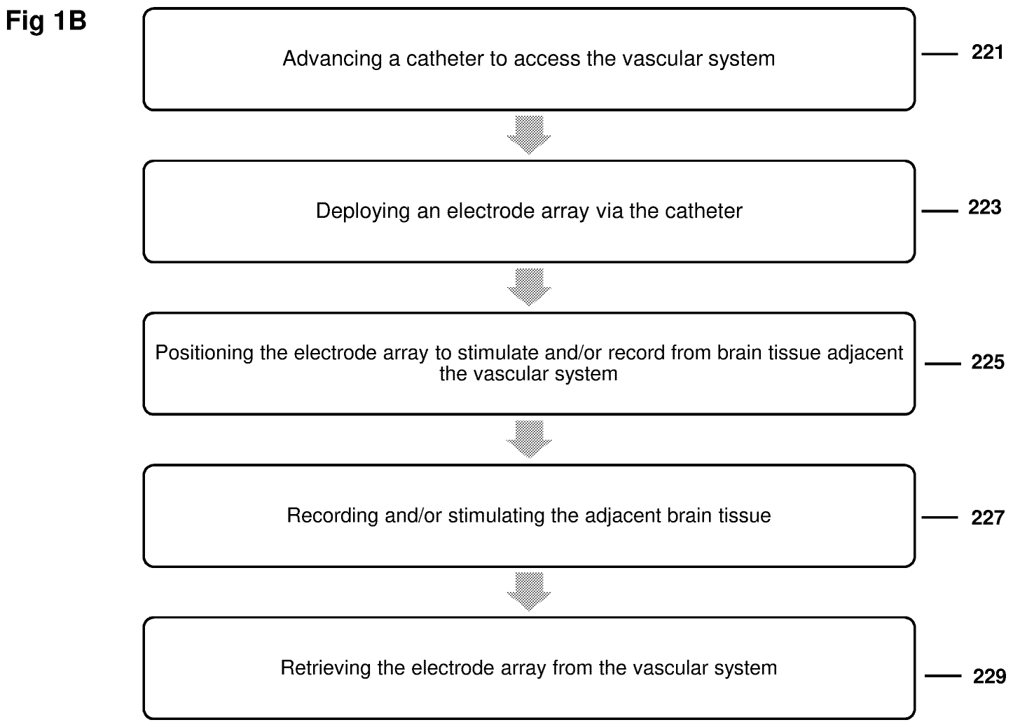

FIG. 1A provides a schematic illustration of an endovascular EEG/ECoG system. As illustrated, the human body has anatomical structures including a brain 101, internal jugular vein 103, subclavian veins 105, superior vena cava 107, inferior vena cava 109, external lilac vein 111, and femoral veins 113.

An endovascular EEG/ECoG system may include an electrode array 201 configured to be positioned within a brain 101. In some embodiments, the electrode array 201 may be positioned in intracranial veins adjacent to the temporal lobe. The electrode array 201 may be connected to a wired connector 203. The wired connector may be configured to pass through the internal jugular vein 103, subclavian veins 105, superior vena cava 107, inferior vena cava 109, iliac vein 111, and/or femoral veins 113. Further, the wired connector 203 may be configured to pass through the axillary, basilic and/or cephalic veins.

The wired connector 203 may connect using a transcutaneous connector to an externally wearable unit 205. Alternatively, the wired connector 203 may connect using a subcutaneous connector to a subcutaneously implanted unit 207.

As illustrated in FIG. 1A, the electrode array 201 may be positioned in the brain 101 using a guiding catheter 301, and introducer sheath 303 assembly 305.

The electrode array 201 may include a stent that is expandable and/or retractable, wire electrodes and/or catheter electrodes.

In some embodiments, the electrode array 201 may include a stent having a scaffold and one or more electrodes positioned on the scaffold. The force of the stent may be calibrated, in that the stent may be designed with a calibrated expansile force so as not to damage or rupture blood vessels when deployed. The stent must have enough expansile force to open completely and appose itself to the blood vessel walls, yet not so much force as to do damage.

In some embodiments the electrode array 201 may include a grid-like array with 4-8 electrodes having one wire per electrode. In some embodiments the electrode array may include a grid-like or irregular array with 8-256 electrodes. In some embodiments, the electrode array 201 may include a grid-like array having hundreds or thousands of electrodes (5-10 micron diameter electrodes, 10-200 micron diameter electrodes, or other sizes) multiplexed for efficient data transfer from the array to an external recording system.

Electrodes may be configured for recording and/or stimulation. In some embodiments, the endovascular stents may be made of nitinol, polymer and/or Polyether ether ketone (PEEK). In some embodiments the endovascular stent (or scaffold) may be made of coated Nitinol. Various techniques can be used for the geometric shape and the deployment system. Geometric shape can be based on any of various self-expanding stent geometries, including closed-cell, open-cell, or hybrid designs. Deployment systems can take into account a need for retrievability. In some embodiments, the endovascular stents may have unconstrained diameter between about 3-10 mm, a length between about 30-70 mm, and the like.

In some embodiments, the endovascular stents may be manufactured from laser-cut PEEK (or other polymer) in order to be electrically insulating. In such an embodiment the use of PEEK stents may insulate electrodes from one another and from other parts of the scaffold itself. Further, in some embodiments, a PEEK laser-cut stent may include metallic electrodes. Metallic electrodes may include gold, silver, platinum, platinum-iridium and the like.

Metallic electrodes may be printed onto or deposited onto a PEEK or other polymer substrate by photolithography, etching, or other bonding processes. In some embodiments, electrodes may be 10-500 microns in diameter. In some embodiments, electrodes may be circular disks and/or square in shape. In some embodiments, the metallic electrodes may be coated to yield optimized recording electrodes. Example coatings include PEDOT and the like.

In some embodiments, PEEK may be laser cut to form a flat surface, and then electrodes may be deposited using lithography or other processes onto the PEEK surface while it is flat. Then the PEEK surface may be wrapped around a mandrel to form a cylindrical stent.

In some embodiments, the electrode array 201 may include a self-expanding stent. The self-expanding stent may be metallic (e.g., Nitinol) or nonmetallic (e.g., PEEK), using a closed-cell design, laser-cut into a closed-cell geometry that would yield a self-expanding stent with the ability to be resheathed and redeployed multiple times, both for adjustment and eventual recapture and removal. The laser-cutting can be performed with the stent-to-be as a flat sheet, which is later wrapped around a mandrel to provide cylindrical form. This has the advantage of permitting electrode deposition on a flat surface, prior to formation of the cylinder. Alternative methods in which the stent is cut from a tube or cylinder are also possible.

Other geometric shapes for self-expanding stents are envisioned, including closed-cell, open-cell, and/or hybrid designs.

In some embodiments, the metallic electrodes may be arranged in a cylindrically symmetrical gird array configuration because rotational orientation within the blood vessel can sometimes be difficult to ascertain, and so the array is agnostic to the degree of rotation of the device within the vessel.

In some embodiments, electrodes may be composed of gold, silver, platinum, or platinum-iridium. In some embodiments, the electrodes are fabricated on the scaffold using lithography, 3D printing, and/or covalent-type bonding processes. Exemplary sizes and shapes of electrodes are discs 25-500 microns in diameter, with impedances in the 1 kOhm range. Alternatively, impedances may be in the range of 25 kOhm.

The wired connector 203 may include one or more lead wires that connect to the electrode array 201. In some embodiments, the wired connector 203 may include fine conductive wire, where each trace is separately insulated and soldered or bonded to electrodes in a one-trace-per-electrode scheme.

In some embodiments, the lead wires may be routed through the delivery catheter assembly 305 through the percutaneous access point in the skin. The lead wires may be left in place after removal of the catheter. In some embodiments the catheter itself, or the wire used to guide the catheter, may be equipped with EEG/ECoG electrodes.

In some embodiments, in the absence of a multiplexer, each electrode may be connected to one wire. In embodiments, where the electrode array/scaffold is a stent, these wires are therefore soldered or bonded to the electrodes on the stent. In embodiments in which the electrode array/scaffold itself is a catheter, the electrodes are exposed on the catheter surface, and the wires are embedded in the catheter walls, with each wire separately insulated.

In embodiments with a multiplexing element present, inputs may be received from each electrode locally (at the catheter tip, for example, or on the stent itself), so that while multiple short electrical connections (lithographically patterned, wired, or other) between electrodes and multiplexer are required, only a limited number of wires (many fewer than the total number of electrodes) must run the length of the delivery catheter extending through the vascular system to the electrode array.

The wired connector 203 may include very small caliber, separately insulated lead wires. In embodiments without a multiplexer, each wire connects to a single electrode. In embodiments without a multiplexer all amplification and signal conditioning is performed external to the body, for example by connecting the lead wires to a conventional commercially available clinical grade EEG/ECoG system.

In embodiments including a multiplexer, the device may include on-board amplification and analog-to-digital conversion. In some embodiments, the device may include EEG/ECoG amplifiers, and the sampling rate and digitization bits could be variable but likely no fewer than 10 bits of digitization and no slower than 20 Hz sampling rate per channel to be clinically useful (in practice much higher sampling rates may be required, up to several hundred Hz or higher).

In some embodiments, the wired connector 203 may connect with an externally wearable unit using soldering and/or bonding between lead wires and power/data electronics. In such an embodiment, leads may be tunneled to a subclavian venous access port. Alternatively, the wired connector 203 may be configured to transcutaneously connect to an external wearable computer 207. The external wearable computer may include a power source, data processing unit, and the like. The external wearable computer may be configured to be “worn” by the patient (e.g., secured to the outside of the chest wall using a sterile adhesive patch). The wires connecting the external wearable computer may be tunneled transcutaneously through the skin, from the endovascular array to the computing unit. In some embodiments, a transcutaneous access port may include a transcutaneous connection and a soft tissue anchor.

In some embodiments, the disclosed systems may utilize venous access techniques common for tunneled peripherally inserted central catheters (PICC) or as used during placement of cardiac devices. The transcutaneous connector may include insulated lead wires passing through the skin, with some additional structural support or coating.

In some embodiments, an endovascular EEG/ECoG system may include an electrode array configured for intravenous use, and a wired connector traversing from the electrode array to an interfacing connector. The wired connector may include circuits or electronics for multiplexing. The interfacing connector may comprise a transcutaneous connector configured to connect to an external wearable unit. Alternatively, the interfacing connector may comprise a subcutaneous connector that forms a subcutaneous implanted unit. The transcutaneous connector or the subcutaneous connector may then interface with a wearable computer configured to provide power, record data, control the operation of the electrode array, and the like.

In some embodiments, external wearable unit 205 and/or external wearable computer 207 may include software for recording EEG/ECoG data. Recording software may be configured to record continuously. The external wearable unit 205 or external wearable computer 207 may comprise a base platform (transcutaneous connector to chest-worn unit for outpatient ambulatory EEG/ECoG, or subcutaneous implant), and platform technology for variety of location-specific endovascular electrodes. The base platform may include a computer for control and data storage for recording and/or stimulation, as well as wireless control and data transfer. This base platform is part of a “modular” system design, and could be used for any of the endovascular electrode systems described herein.

As the leads from the recording electrodes exit the brain, they form a bundle that is tunneled through a subcutaneous layer to a microcomputer or other device designed to power the electrode system, store recording data, store stimulation parameters and other parameters when applicable, and coordinate wireless data telemetry with external devices, among other functions. These active electronic components are contained within the hermetic package. In such a configuration, the electrode array permits long-term electroencephalographic or electrocorticographic monitoring of patients in the ambulatory setting, as there is no fluidic communication between the brain and the outside world, and hence no major risk of intracranial infection. In this configuration, the monitoring capabilities of the minimally invasive system disclosed here offer an option not available using conventional grid and strip (EcoG) electrodes, which are implanted via craniotomy, tunneled through dura, skull, and skin, and permit leakage of cerebrospinal fluid and a conduit between the brain and the outside world. Epilepsy patients undergoing monitoring using such techniques, which represent the present state of the art, must be monitored in a hospital setting until the recording electrodes are removed. Furthermore, in the current state of the art, removal of the electrodes requires a second operation for electrode removal, and sometimes also for repair of the dura membrane and reaffixing of the removed portion of the skull.

Angiographic techniques may be used for the placement of the electrode array 201 within the brain 101. For example, in some embodiments, the electrode array 201 may be delivered through an endovascular catheter navigated over an endovascular wire. In other embodiments, the catheter or wire itself may contain embedded electrodes from which recordings and/or stimulation could be performed. In some embodiments, these recordings could be made in an exploratory fashion prior to any permanent or longer-term electrode array placement.

In some embodiments, the electrode array 201 may have a closed-cell stent design. For example, the stents may be deployed, resheathed, and re-deployed if repositioning is necessary.

In some embodiments, electrode arrays 201 may be removed using a catheter-based recapture system. In some embodiments the electrode arrays 201 may require wired connections to the recording electronics. These same wire connections may operate to guide a catheter (catheter-over-wire) back to the position of the stent. The stent can then be recaptured into the catheter by pulling the recording wires so as to resheath the stent within the catheter, which can then be removed from the body.

In some embodiments, a catheter may be inserted into the femoral artery and guided to an artery in the neck. A variety of polymer materials and coatings are used to produce endovascular catheters. For example, endovascular catheters may use nylon, polyurethane, polyethylene terephthalate (PET), latex, thermoplastic elastomers, polyimides, and the like. Further, some endovascular catheters may include thin hydrophilic surface coatings.

In some embodiments, the disclosed systems may be used for stimulation. For example, stimulation parameters may be tested in a supervised procedure. In other embodiments, stimulation may be delivered through catheter and stent electrodes are the like for intermittent stimulation. For example, stimulation may be applied at a frequency of 60 Hz, square wave, charge-balanced waveform, having an amplitude of 0.1 to 20 mA. In the case of a device implanted for a period of time (as in ambulatory EEG/ECoG), stimulation parameters and stimulation schemes may resemble those by deep brain stimulators, spinal cord stimulators and/or responsive stimulation systems.

FIG. 1B is a flowchart illustrating a method in accordance with embodiments of the present disclosure. As illustrated, in a first step, a catheter may be advanced to access the endovascular system 221. In a second step, an electrode array may be deployed via the catheter 223. In a third step, the electrode array may be positioned to stimulate and/or record from brain tissue adjacent to the endovascular system 225. In a fourth step, electrical signals may be recorded from or applied to (i.e., stimulating) the adjacent brain tissue 227. In a fifth step, the electrode array may be retrieved from the endovascular system 229.

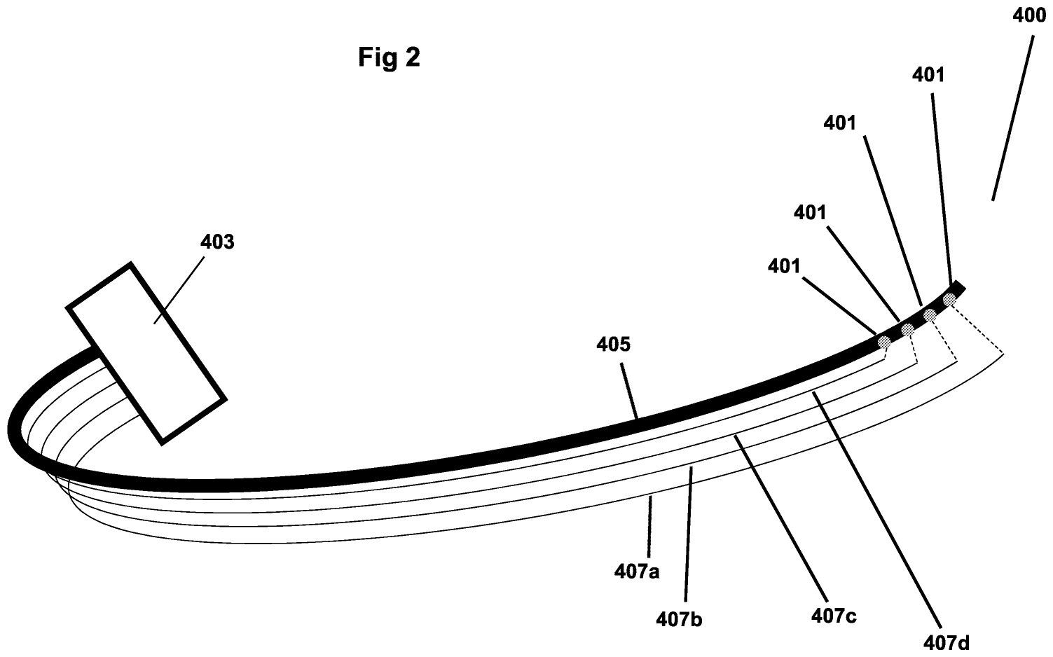

FIG. 2 illustrates an electrode array 400 built in accordance with the present disclosure. The depicted electrode array includes four electrodes 401 positioned at a first end of the electrode array 400. Each electrode may be connected to an amplifier and recording apparatus 403 located at a distance from the electrodes. Traces to each electrode 407a, 407b, 407c, 407d are separately insulated. In some embodiments, the traces 407a, 407b, 407c, and 407d may be bundled together as a single composite “wire” 405 which is coated with insulated and hydrophilic coatings (as described above with respect to wire connector 203).

In some embodiments the composite “wire” 405 may be 8-35 thousandths of an inch in diameter and have a length of approximately one meter. Its diameter may taper toward the distal tip. In some embodiments such a composite “electrode wire” could have many more than four electrodes.

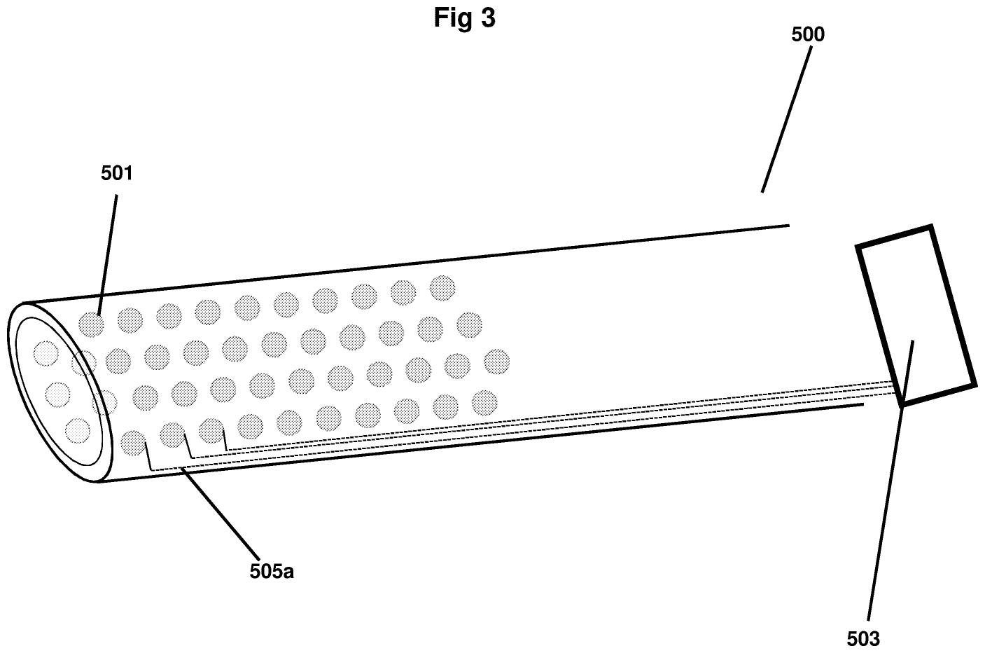

FIG. 3 illustrates an electrode array 500, where the electrode array 500 is shaped as a catheter. The electrode array 500 may include a plurality of distal electrodes 501. Each electrode may be connected via a lead wire 505 to an amplifier and recording apparatus 503. In some embodiments, each lead wire 505 may be separately insulated and embedded within the wall of the catheter. The lead wire may be configured to be exposed only at the point of contact with the electrode and where it connects to the amplifier and recording apparatus 503 (outside of the body). In some embodiments, the catheter shaped electrode array 500 may be less than 3 mm in diameter and span approximately 1 meter in length.

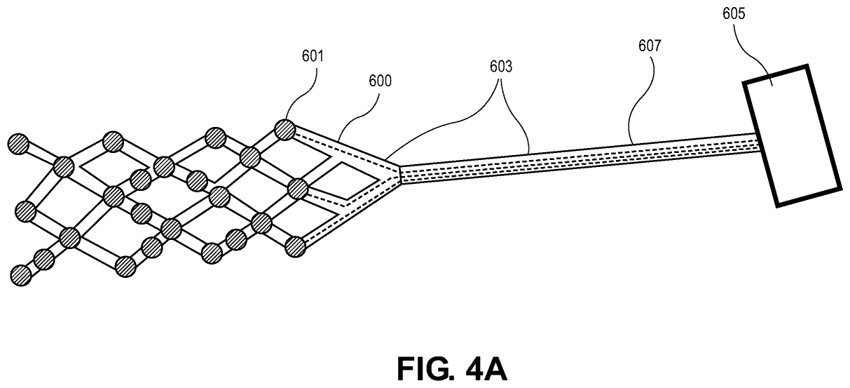

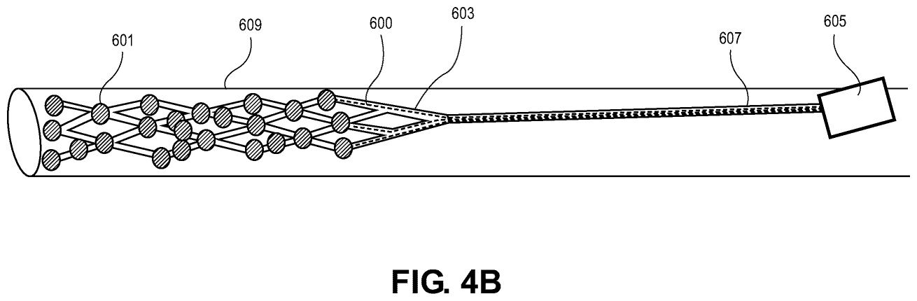

FIGS. 4A and 4B illustrate an endovascular electrode array that has a collapsible structure. As illustrated the array 600 may include a plurality of electrodes 601 positioned along the collapsible structure. As illustrated in FIG. 4A the array may be expanded while recording and/or stimulating. As illustrated in FIG. 4B, the array may be collapsed into a catheter. Each of the electrodes 601 may be connected to a lead wire 603. Electrodes 601 may be insulated from one another and from the lead wires. Further, the lead wires 603 may be insulated from one another. Additionally, in some embodiments, the array 600 may be made of insulating materials including polymers such as PEEK. As illustrated, each lead wire 603 may be separately insulated and only exposed at the point of contact with the electrode (distally) 601 and where exposed to amplifier and recording apparatus 605 (proximally, outside the body).

In some embodiments, a stent control wire or microcatheter 607 may be used for stent delivery. In some embodiments, the lead wires 603 may be bundled together along the stent control wire 607. In some embodiments, the wire traces may extend for a length of one meter or more. The stent 600 may be approximately 3-5 cm in length. As illustrated in FIG. 4B, the stent scaffolding 600 can be collapsed to fit within a delivery catheter 609. While the stent scaffolding 600 folds (elongating as it collapses), the electrodes 601 remain the same size.

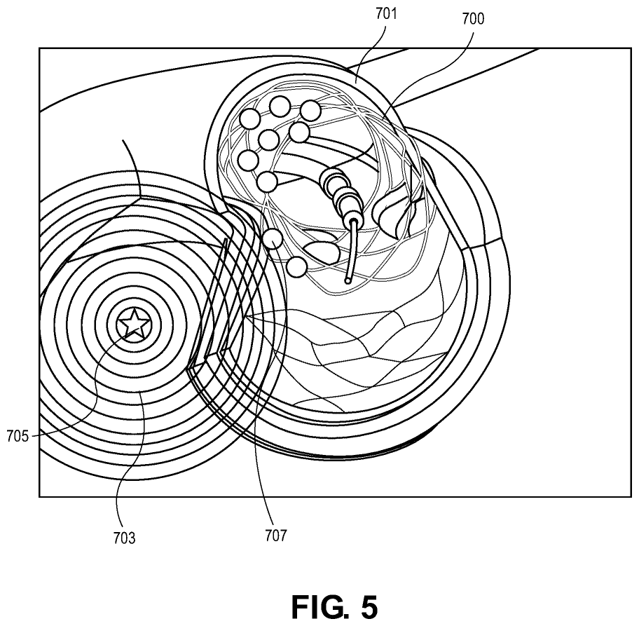

FIG. 5 illustrates a schematic for endovascular approaches. As illustrated, a stent electrode array 700 is deployed within a blood vessel 701 which is located adjacent to brain tissue 703. A portion of the brain tissue associated with epileptogenic focus may be emitting abnormal electrical activity 705. The abnormal electrical activity may be recorded by an electrode 707 located on the stent electrode array 700, or by multiple adjacent electrodes in a manner that permits both spatial and temporal localization of the neural activity.

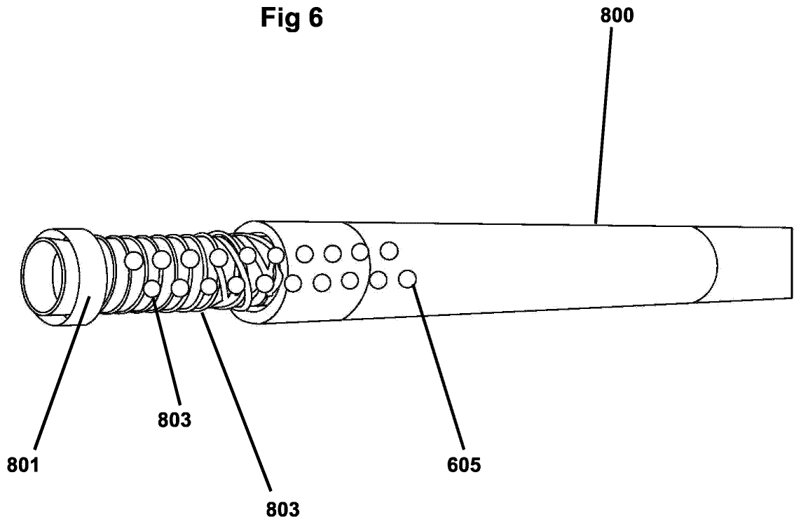

FIG. 6 illustrates an example of a catheter based electrode array 800. As illustrated a catheter may include a radio-opaque tip marker 801 that may be adapted to be an electrode and connected to the inner braiding 803. The catheter may include braided wires 803 that are used for mechanical support. The braided wires 803 may be individually insulated such that only a proximal end (configured to connect to an amplifier and recording system) was exposed and a small region at the distal end, tip of the catheter was also exposed. Electrodes 805 may be positioned along the braided wires. In some embodiments, the catheter may be electrically insulated with polymer with hydrophilic coating. Examples of electrode materials include gold, silver, platinum, and the like.

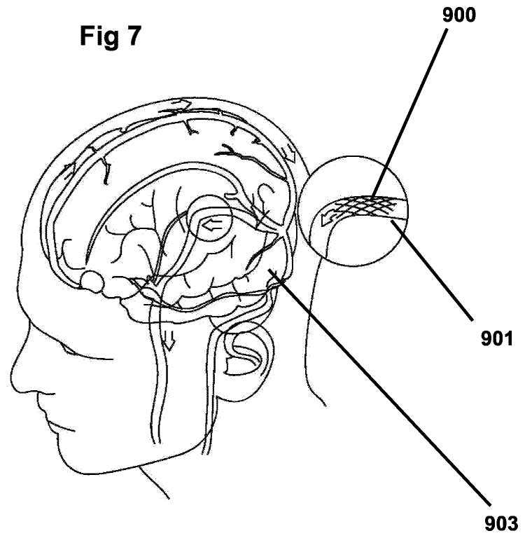

FIG. 7 illustrates an example of an electrode array built in accordance with embodiments of the present disclosure positioned within a blood vessel. As shown, an electrode array (i.e., stent-based electrode array) may be delivered to a blood vessel 901 located within the brain 903. The stent may be positioned adjacent to a brain target of interest. For example, in some embodiments, the stent may be a self-expandable stent, that is advanced to through the vascular system into a blood vessel within the brain. The stent may then be expanded and deployed, such that the electrodes positioned within the stent are able to record from the surrounding brain tissue. Further, in some embodiments, after recordings are obtained, the stent may be collapsed, retrieved, and removed from the body via the endovascular system. In some embodiments, the stent may have an unconstrained diameter between about 3-10 mm and a length between 30-40 mm.

Embodiments related to the present disclosure include endovascular (venous or arterial) electroencephalography (EEG/ECoG) electrode arrays and related systems. These include electrode arrays designed for deployment in the blood vessels of the brain for neural recording, stimulation, or both. Specific designs include inferior petrosal sinus and cavernous sinus (venous) electrodes for neural interfaces and electroencephalography/electrocorticography. Embodiments built in accordance with the present disclosure may be used for electrophysiological “mapping” of cortical (especially deep cortical) regions such as the temporal lobe from anterior, posterior, medial, lateral, superior, and inferior locations. An endovascular EEG/ECoG device may be shaped as a catheter, microwire, stent, or other configuration implanted, for example, in the inferior petrosal and cavernous venous sinuses. Access is possible, for example, via the femoral, axillary, basilic, cephalic, subclavian, or other veins. Transcutaneous connectors (leads or electrodes) to external wearable devices are envisioned.

Embodiments built in accordance with the present disclosure may allow for the ability to perform dynamic, real-time mapping of brain electrical activity by navigating electrode arrays through the blood vessels using techniques borrowed from conventional neuro-angiography. Conventional systems are unable to perform dynamic, three-dimensional mapping techniques of this nature; instead, the conventional systems use effectively static electrode arrays.

In some embodiments, the disclosed endovascular electroencephalography (EEG) or electrocorticography (ECoG) electrode arrays may be used to record for approximately days in an ambulatory or outpatient context. In such a system, continuous electroencephalographic (EEG) or electrocorticographic (ECoG) recording may be performed in the ambulatory setting, wherein the recording electrodes are located within the blood vessels of the brain (particularly the veins). The ambulatory EEG/ECoG system may include leads that connect to the endovascular electrodes which pass through the vascular system, then exit the blood vessels to pass through the subcutaneous tissues, and either tunnel transcutaneously to a device worn on the external surface of the body, or tunnel subcutaneously to a similar device implanted in the subcutaneous tissues. Accordingly, the disclosed systems may provide medium-term (days, weeks) continuous EEG/ECoG recording to detect, characterize, and localize the onset of seizure activity.

Embodiments built in accordance with the present disclosure may be configured for performing continuous electroencephalographic recording in the ambulatory setting as described, wherein the recording electrodes are located within the blood vessels of the brain. The disclosed system further comprises leads that connect to the endovascular electrodes which pass through the venous system, then exit the venous system to pass through the subcutaneous tissues, and either tunnel transcutaneously to a device worn on the external surface of the body, or tunnel subcutaneously to a similar device implanted in the subcutaneous tissues. Intravenous targets may include the dural venous sinuses, inferior petrosal sinus, and/or the cavernous sinus, as well as deep veins and superficial cortical veins.

In some embodiments, the disclosed devices may be inserted into the cerebral veins via a peripheral vein of the upper extremity (basilic vein, brachial vein, cephalic vein, subclavian vein) or via a peripheral vein of the lower extremity (external iliac vein, common femoral vein) or via a central venous catheter to veins such as the internal jugular vein in the neck.

The devices may be delivered using interventional techniques via a 1-5 mm incision at the venous puncture site. The devices can be delivered in an outpatient setting and the patient can be discharged to home on the same day. The devices may be positioned at various locations in the cerebral venous system, according to the clinical scenario. Possible locations for recording in the venous system include the cerebral venous sinuses (superior sagittal dural venous sinus, straight dural venous sinus, lateral dural venous sinus) and veins of the skull base (cavernous sinus, inferior petrosal sinus), as well as deep veins and superficial cortical veins.

The disclosed devices can be placed for variable durations according to the clinical scenario. The recording can last from several seconds to minutes to 1 hour to 30 days depending on the clinical scenario. At the end of the recording period, the device may be removed via a minimally invasive approach.

Intra-arterial targets may include the internal carotid, and/or the external carotid, and branches of those arteries. For example, the disclosed devices may be inserted via a peripheral artery in the upper extremity (radial, ulnar, brachial arteries) or the lower extremity (iliac, femoral arteries). Similar to the procedure discussed above with regards to venous puncture sites, with respect to an intra-arterial target, the disclosed devices may also be delivered using interventional techniques via a 1-5 mm incision at the arterial puncture site, such that the devices can be delivered in an outpatient setting and the patient can be discharged to home on the same day. The devices will be positioned various locations in the cerebral arterial system, according to the clinical scenario. Possible locations for recording in the arterial system include the internal carotid arteries and their branches (anterior and middle cerebral arteries) the basilar artery and its branches (superior cerebellar and posterior cerebral arteries) and the external carotid artery and its branches (internal maxillary artery, middle meningeal artery, superficial temporal artery).

The devices can be placed in the arterial system for a short duration (up to 5 hours) as prolonged duration of these devices in the cerebral arterial system carries risk of thromboembolic complications (i.e. stroke), though the risk of such complications can be minimized when electrodes are delivered using catheters though which anticoagulant (“blood-thinning”) solutions are infused during the procedure, as is standard practice in many angiographic procedures. When the electrode array is itself mounted on a catheter, this scheme is particularly straightforward to implement, though it is also possible to implement when the electrode array is based on a stent or other endovascular structure.

Endovascular techniques can provide advantages over many conventional systems. For example, large arteries and veins are located in proximity to the brain structures involved in epilepsy. The temporal lobe and the frontal lobes are the most common parts of the brain involved in generating seizures and causing epilepsy. Via the endovascular approach, recording devices can be placed along the surfaces of or within the depths of these regions. While the endovascular approach allows recording from the surface of the brain, the placement of the recording device is via a minimally invasive approach without the risks and hazards of open brain surgery. The placement of a device via the endovascular approach can be done in an outpatient/ambulatory setting. Via the endovascular approach, devices may be placed in deep structures of the brain, inaccessible even with open surgery. Intravenous approaches will allow for placement of one or more devices for prolonged recording up to 30 days. Further, the intravenous approach (in contrast with some arterial approaches) does not significantly raise the risk of stroke. The endovascular techniques described herein can reach more and deeper areas of the brain compared to surgical implantation of electrodes, with a less invasive approach for longer durations.

In some embodiments, systems and methods built in accordance with the present disclosure may include endovascular neural stimulating electrodes that may be used for medium- to long-term applications. For example, the disclosed systems and methods may be used for testing stimulating for microvascular compression syndromes (for trigeminal neuralgia, hemifacial spasm, and other possible neurovascular compression syndromes), to confirm diagnosis prior to surgical intervention and also for vascular exploration to find vascular compression points prior to surgery for microvascular decompression. Additional therapeutic stimulation technologies may be developed.

Further, stimulating electrodes may be used in spinal radicular arteries to identify radicular pain distribution, location of nerve compression, and guide therapy (surgical/endoscopic decompression, epidural stimulation or injections).

### Applications to Treatment of Epilepsy

The disclosed systems and methods may be used for the detection and/or treatment of epilepsy. Fifty million people in the world have epilepsy, and there are between 16 and 51 cases of new-onset epilepsy per 100,000 people every year. A community-based study in southern France estimated that up to 22.5% have drug resistant epilepsy. Patients with drug-resistant epilepsy have increased risks of premature death, injuries, psychosocial dysfunction, and reduced quality of life.

Approximately three million American adults reported active epilepsy in 2015. Active epilepsy, especially when seizures are uncontrolled, poses substantial burdens because of somatic, neurologic, and mental health comorbidity; cognitive and physical dysfunction; side effects of anti-seizure medications; higher injury and mortality rates; poorer quality of life; and increased financial cost. The number of adults reporting that they have active epilepsy significantly increased from 2010 (2.3 million) to 2015 (3 million), with about 724,000 more cases identified from 2013 to 2015. An estimated 20-30% of patients with epilepsy have medically and socially disabling seizure disorder which leads to increased morbidity and mortality, depression and physical trauma.

“Medically intractable” patients by definition have failed at least two antiepileptic medications. The chance of becoming seizure free after failing two appropriate seizure medications is extremely low. Severe medication side effects may also be an indication for surgery. To determine if a patient is a candidate for epilepsy surgery, an extensive evaluation is undertaken, including testing modalities such as video EEG telemetry, anatomical (MM) and functional (positron emission tomography (PET) or single photon emission computerized tomography (SPECT) imaging), endovascular-assisted pharmacologic assessment (“Wada” testing), neuropsychological testing, electrocorticography (ECoG) and depth electrode mapping.

Conventional EEG is an important diagnostic test in the evaluation of a patient with epilepsy. During a conventional EEG test, electrical activity is recorded from standard sites on the scalp according to the standard 10-20 system of electrode placement. The EEG recording depends upon differential amplification between paired inputs, each pair of inputs generating a single output channel, with data readout in the form of a voltage tracing. Despite the widespread availability and ease of usage of EEG testing there two major limitations: (1) intermittent EEG changes reflecting abnormal (seizure) activity can be infrequent and may not appear during the period of recording which may range from 30 minutes to 3 days, and (2) some highly epileptogenic areas, such as the medial temporal lobes, are not well explored by the scalp electrodes and so the diagnostic yield is suboptimal.

In an alternative to conventional EEG, other electrodes have been developed to engage with the sphenoidal, nasopharyngeal, ear canal, and/or mandibular notch, in order to aid with the diagnosis of seizures. However, these alternatives are often uncomfortable to the patient and prone to artifacts and misinterpretation, providing limited usage and yield in practice.

For patients requiring more invasive evaluation, conventional practice involves the use of intracranial EEG in the form of electrocorticography (ECoG) or multiple depth electrode placement (“stereo-EEG” or sEEG). This approach requires surgical implantation of EEG electrodes in order to better lateralize and localize seizure foci. Electrodes placed on the brain surface and directly in the brain can be used to map seizure activity. Placement of these electrodes requires craniotomy (or at least placement of multiple burr holes through the skull) for surgical implantation, while the patient needs to remain hospitalized for 3-5 days while the electrodes are recording. Then, a second surgery is necessary to remove the electrodes and restore the craniotomy defect. However, it is possible that even after the surgical implantation, the location of a single seizure focus is not determined. The invasive nature of these procedures and the possible failure to identify and localize seizure origin indicate a need for more accurate and less invasive means of identifying and localizing seizure foci.

In contrast, embodiments built in accordance with the present disclosure provide minimally invasive alternatives for patients with epilepsy who require evaluation for surgery. Currently, implantation of electrodes on surface of the brain prior to definitive surgery to remove a seizure leads to a requirement of two open cranial surgeries and prolonged hospital stays. As a result, many patients are reluctant to undergo such evaluations. By contrast, the disclosed systems and methods may provide minimally invasive alternatives that will allow for implantation of diagnostic electrodes for up to 30 days, without the need for cranial surgery. The disclosed embodiments may allow a safe and minimally invasive option for recording from the surface and deep structures of the brain.

Embodiments built in accordance with the present disclosure may allow for inpatient as well as outpatient recordings using an endovascular approach. In some embodiments, intra-procedural endovascular recordings may provide an immediate advantage over conventional EEG and related recording modalities because the endovascular (or angiographic) nature of the procedure will permit dynamic electrophysiologic exploration of the brain in three-dimensions, which is not possible with any existing technology.

Further, in some embodiments, the systems and methods described herein will allow patients to benefit from a minimally invasive approach. Additionally, the described embodiments may allow recordings that are multiple days in duration, without the requirement that patients be admitted to stay in the hospital for the duration of the recording.

Such procedures will be useful not only for patients contemplating surgery, but also to determine whether a patient who is responsive to medical management might be safely trialed on a different dose, different medication, or taken off medications altogether without experiencing a seizure. Existing invasive mapping procedures are not useful to medically managed patients because the risk of an invasive procedure is not typically worth the potential benefit of a change in medications. However, because many medications have undesirable side effects the possibility of such a minimally invasive procedure can potentially benefit even patients who are not considering surgical treatments of their epilepsy. Patients and physicians may want the security of adjusting medications or dosages while continuously recording EEG in an ambulatory context in accordance with the systems and methods described herein.

Additionally, nonconvulsive seizures (NCS) and nonconvulsive status epilepticus (NC SE) are neurological emergencies that occur in critically ill patients, and they are seen more frequently in patients with acute or chronic neurologic injury (stroke, trauma). Previous retrospective and prospective studies have shown the prevalence rate of seizures in neurologic intensive care units to be 8% to 48%. Because routine EEGs detect less than 50% of seizures that will eventually be noted in critically ill patients, a routine EEG is often not sufficient to rule out seizures in patients admitted to the intensive care unit. Thus, patients having NCS and/or NCSE are often too ill to undergo surgical implantation of electrodes, and due to the limitations of surface EEG they remain undiagnosed and suboptimally treated. Thus, these patient populations would also benefit from the systems and methods described herein. The endovascular EEG/ECoG systems and methods described herein may provide the ability for minimally invasive EEG/ECoG recordings from the surface and the deep parts of the brain and aid in the diagnosis and management of this group of patients.

In some embodiments, the disclosed electrode arrays may be configured to perform mapping procedures in the context of temporal lobe epilepsy (TLE). In such an embodiment, the disclosed systems and methods may be used to electrophysiologically localize and stimulate targets within wide regions deep within the brain.

Conventional ambulatory EEG systems are configured to record electrical activity produced by the brain as a patient goes about his or her normal routine. Patients are fitted with multiple scalp electrodes (e.g., anywhere from 16 to 24 to potentially many more) in place for several days. For that reason, ambulatory EEGs are quite restrictive in practical terms with respect to what patients are able to do, as they are bulky and cumbersome. Accordingly, ambulatory EEG systems are not widely used. However, typical ambulatory EEGs do not require any surgery, and the scalp electrodes are secured to the patient with adhesives. Conventional systems are unable to provide ambulatory ECoG system in current clinical use, as existing methods for safely maintaining ECoG electrodes require intensive monitoring of patients in supervised, inpatient settings. The systems and methods of the present disclosure provide for the possibility of safe, effective ambulatory ECoG.

The present disclosure provides systems and methods for developing electrode arrays that can be deployed within a patient's brain using minimally invasive surgical techniques, causing minimal to no collateral damage to normal brain tissue. The disclosed arrays can be manipulated in dynamic, exploratory ways during and after deployment in order to achieve optimal recording performance and test electrophysiologic hypotheses regarding the precise location of abnormal brain activity. The arrays may be optimized for recording, stimulation, or both functions. Further, the disclosed arrays may provide excellent spatial and temporal resolution due to the optimized properties of the electrode contacts.

Advantages over Prior Techniques

In some embodiments, the disclosed systems and methods utilize an endovascular approach, in that the disclosed systems may deploy electrodes within the blood vessels and/or cavities of the brain. Conventional systems are unable to utilize an endovascular approach, due in fact to the anatomical constraints of the vascular system (e.g., size, positioning), the risks associated with operating in the vascular system (e.g., obstruction of flow), and the like. Conventional systems have also been limited by materials, fabrication techniques and electronic technology.

In some embodiments, the disclosed systems and methods may be used to perform electrical recordings from the brain and nervous system. In some embodiments, the disclosed systems and methods may be used to stimulate certain regions of the brain and nervous system. Electrodes may be placed in minimally invasive fashion within the blood vessels, arteries and veins of the brain, head, and neck. The technologies described herein relate to the designs of electrode arrays for deployment in specific endovascular anatomic locations, mechanical systems for stabilizing such electrode arrays, systems for delivering such electrode arrays to endovascular targets, systems for retrieving such electrode arrays following deployment, and systems for communicating with such electrode arrays while they are deployed.

The systems and methods disclosed herein expand upon conventional neuro-angiographic (i.e., “interventional neuroradiology,” “endovascular neurosurgery”) techniques. The disclosed systems may include endovascular catheters, wires, stents, scaffolding and the like. The disclosed systems and methods are configured to access the blood vessels of the brain using wires and catheters that can be navigated in controlled fashion, using image-guidance, through the blood vessels of the brain in either exploratory or precise deterministic ways.

In comparison to conventional systems and methods, the disclosed technologies may be deployed using minimally invasive surgical techniques, causing minimal to no collateral damage to normal brain tissue. By contrast, conventional electrodes may require highly invasive procedures for implantation, and/or they may damage areas of the brain surrounding the areas where the electrodes are placed.

Additionally, in comparison to conventional systems and methods, the disclosed technologies may include electrode arrays that can be manipulated (i.e., repositioned) in dynamic, exploratory ways during and after deployment. This allows for optimal recording performance and testing of electrophysiologic hypotheses regarding the precise location of abnormal brain activity. By contrast, conventional arrays for depth recording cannot realistically be moved in dynamic fashion, apart from small adjustments to depth at the time of initial placement. The disclosed arrays can be optimized for recording, stimulation, or both functions, and they provide excellent spatial and temporal resolution due to the optimized properties of the electrode contacts.

In some embodiments, systems and methods in accordance with the present disclosure may be used for performing continuous electroencephalographic recording in the ambulatory setting. In such a setting recording electrodes may be located within the veins of the brain. In some embodiments, the system further includes leads that connect to the endovascular electrodes which pass through the vascular system (especially the venous system), then exit a blood vessel to pass through the subcutaneous tissues, and either tunnel transcutaneously to a device worn on the external surface of the body, or tunnel subcutaneously to a similar device implanted in the subcutaneous tissues, and/or the like.

The disclosed systems and methods may be capable of providing medium term (i.e., days, weeks) of continuous EEG/ECoG recording in order to detect, characterize and localize the onset of seizure activity. Accordingly, the disclosed systems and methods may provide a useful tool for focal epilepsy.

While the disclosure has been described in connection with certain embodiments, it is to be understood that the disclosure is not to be limited to the disclosed embodiments but, on the contrary, is intended to cover various modifications and equivalent arrangements included within the spirit and scope of the disclosure, which scope is to be accorded the broadest interpretation so as to encompass all such modifications and equivalent structures as is permitted under the law.

1. An implantable medical device comprising:
an amplifier and recording apparatus; and
a composite wire bundle comprising a plurality of electrodes and a plurality of separately insulated traces connecting the amplifier and recording apparatus to the plurality of electrodes, wherein the plurality of electrodes are arranged linearly with respect to each other along the composite wire bundle, wherein each of the separately insulated traces comprises a different length, wherein the composite wire bundle tapers towards a distal tip thereof, and each of the plurality of electrodes is configured to at least one of stimulate or record from neural tissue.

2. The implantable medical device of claim 1, wherein the composite wire bundle has a diameter between 8 and 35 thousandths of an inch.

3. The implantable medical device of claim 1, wherein the composite wire bundle has a length of approximately one meter.

4. The implantable medical device of claim 1, wherein the amplifier and recording apparatus comprises an embedded multiplexing unit having an analog-to-digital converter.

5. The implantable medical device of claim 4, wherein the embedded multiplexing unit comprises an on-board amplifier and an analog-to-digital converter.

6. The implantable medical device of claim 1, comprising:
a wired connector configured to receive one or more electrical connections from the amplifier and recording apparatus.

7. The implantable medical device of claim 6, comprising:
a transcutaneous connector configured to connect the wired connector to an externally wearable unit.

8. The implantable medical device of claim 7, comprising:
a subcutaneous connector configured to connect the wired connector to a subcutaneously implanted unit.

9. The implantable medical device of claim 1, wherein each of the plurality of separately insulated traces are soldered or bonded to each of the plurality of electrodes.

10. A method comprising:
positioning an implantable medical device proximate to brain tissue, wherein the implantable medical device comprises an amplifier, a recording apparatus, and a composite wire bundle comprising a plurality of separately insulated traces and a plurality of electrodes, wherein the plurality of electrodes are arranged linearly with respect to each other along the composite wire bundle, wherein each of the separately insulated traces comprise a different length, wherein the composite wire bundle tapers towards a distal tip thereof,
connecting an amplifier and a recording apparatus to the plurality of electrodes with the plurality of separately insulated traces; and
stimulating or recording neural tissue with the plurality of electrodes.

11. The method of claim 10, wherein positioning the implantable medical device proximate to the brain tissue comprises:
adjusting a location of the implantable medical device responsive to recording at least one electrophysiological signal from the brain tissue.

12. The method of claim 10, wherein the composite wire bundle has a diameter between 8 and 35 thousandths of an inch.

13. The method of claim 10, wherein the composite wire bundle has a length of approximately one meter.

14. The method of claim 10, wherein the amplifier and the recording apparatus comprises an embedded multiplexing unit having an analog-to-digital converter.

15. The method of claim 10, comprising:
a wired connector configured to receive one or more electrical connections from the amplifier and the recording apparatus.

16. The method of claim 15, comprising:
a transcutaneous connector configured to connect the wired connector to an externally wearable unit.

17. The method of claim 15, comprising:
a subcutaneous connector configured to connect the wired connector to a subcutaneously implanted unit.

18. The method of claim 10, further comprising:
bonding or soldering each of the plurality of separately insulated traces to each of the plurality of electrodes.
